(19) United States
(12) Patent Application Publication
(10) Pub. No.: US 2009/0264822 A1
(43) Pub. Date: Oct. 22, 2009

(54) Method of Making a Zero-Fold Balloon With Variable Inflation Volume

(75) Inventor: David Johnson, Turloughmore (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/106,707

(22) Filed: Apr. 21, 2008

(51) Int. Cl. A61M 25/10; B29D 22/02

(52) U.S. Cl. 604/103.07; 264/573

(57) ABSTRACT

The present invention provides a method of making a zero-fold dilatation balloon. Typically, the method includes: providing a tubular parison comprising a polymeric material; providing a source of heat and pressure for forming a balloon pre-form; heating, stretching, and expanding the tubular parison to form an expanded parison without confining the size of the expanded parison by a mold wall internal surface; and subjecting the resultant parison to a heat setting process to form a zero-fold balloon having a uniform profile along its entire length in a deflated state.

### BACKGROUND OF THE INVENTION

Minimally invasive intravascular procedures employing balloons and medical devices incorporating those balloons (i.e., balloon catheters) are becoming more common and routine. More particular, low pressure high compliance balloons are important for occlusion and fixation applications where they are required to inflate to relatively large diameters with minimum stress transferred to the target vessel. Such applications include venogram balloon catheters used in pacemaker or defibrillator lead placement for visualizing the coronary veins in advance of placing the lead. Another application is temporary vessel occlusion to contain emboli and facilitate aspiration or irrigation to remove emboli particles and debris within the vessel. With respect to fixation applications, balloons are used on guidewires to anchor and hold the distal portion of the guidewire in a relatively fixed position while catheters, scopes or other instruments are advanced/retracted over the proximal end and/or body of the guidewire. It is further desirable that such anchoring member be incorporated into the guidewire without substantially increasing the diameter of the guidewire and without otherwise interfering with the normal mode of use of such guidewire.

Other procedures include angioplasty procedures that are conducted when it becomes necessary to expand or open narrow or obstructed openings in blood vessels and other passageways in the body to increase the flow through the obstructed areas. For example, in an angioplasty procedure, a dilatation balloon catheter is used to enlarge or open an occluded blood vessel that is partially restricted or obstructed due to the existence of a hardened stenosis or buildup within the vessel. This procedure requires that a balloon catheter be inserted into the patient's body and positioned within the vessel so that the balloon, when inflated, will dilate the site of the obstruction or stenosis so that the obstruction or stenosis is minimized, thereby resulting in increased blood flow through the vessel.

In some instances, the extent of the occlusion is so severe that the vessel is completely or nearly completely obstructed, which may be described as a total occlusion. If this occlusion persists for a longer period of time, the lesion is referred to as a chronic total occlusion or CTO. Total or near-total occlusions in arteries can prevent all or nearly all of the blood flow through the affected arteries. It has been estimated that 5% to 15% of patients on whom percutaneous transluminal coronary angioplasty (PTCA) is attempted are found to have CTOs of at least one coronary artery. In patients who suffer from coronary CTOs, the successful performance of a PTCA is a technical challenge.

Angioplasty balloons are typically tightly folded and wrapped upon themselves for delivery to the targeted lesion, storage, and are unwrapped and expanded to a size that is considerably greater than the stored size by the introduction of an expansion fluid into the balloon. However, the extreme narrowing at a CTO still prevents the passage of a tightly wrapped balloon. The desire to treat such narrowed vessels and also the desire to treat more distal and narrower vessels has led to a desire to reduced the balloon catheter crossing profile by creating a zero fold balloon that ideally has approximately the same diameter as the catheter shaft.

Low-pressure highly compliant elastomeric balloons are typically dip molded from thermosetting elastomers, inflated by volume, and capable of recovering close to their original dimensions. However, the dip molded balloon process is a low volume production process.

Low-pressure balloons can also be stretch-blow molded from thermoplastic elastomers such as thermoplastic polyurethane (TPU), whereby the material is processed to conform to the internal dimensions of a mold. One drawback of stretch-blow molded balloons is that large diameter balloons must be folded or wrapped to satisfy guide catheter compatibility requirements. Another drawback is the bagginess of the fully deflated balloon. A multi-block copolymer developed for use as a zero-fold balloon is described in U.S. Pat. Pub. No. 2005/0118370. However, stretch blow molded large outer diameter balloons with minimum-profile deflation characteristics that can be inflated to various diameters have heretofore been unavailable.

### SUMMARY

The present invention provides a method of making a zero-fold dilatation balloon. Such balloons include a body having a proximal end and a distal end, and comprise a continuous thermoplastic elastomeric polymer tube with a generally uniform profile along its entire length in a deflated state; wherein such balloon can be inflated to various volumes (preferably up to 15 millimeters (mm) in diameter). Preferably, such balloons can be deflated to the original size. The original size is also ideally approximate the catheter shaft diameter on which the balloon is mounted.

In one embodiment, the present invention provides a method of making a dilatation balloon. The method includes: providing a tubular parison comprising a polymeric material; providing a source of heat and pressure for forming a balloon; heating, stretching, and expanding the tubular parison to form an expanded parison without confining the size of the expanded parison by a mold wall internal surface; and subjecting the resultant parison to a heat setting process that is carried out for a time sufficient to form a zero-fold balloon having a uniform profile along its entire length in a deflated state. Preferably, the pressure is reduced during the heat setting step, which causes the parison to retract to the pre-form shape

Although a mold can be used if desired for parison expansion in the methods of the present invention, the resultant parison would not be expanded to contact and be confined with the internal surface of the mold wall such that its size is determined by the mold wall.

In certain embodiments, heating, stretching, and expanding the tubular parison to form an expanded parison comprises axially stretching and radially expanding the tubular parison at a temperature above the Tg of the polymeric material. Such process step is subjected to a pressure sufficient to stretch and thin the balloon walls, preferably at a pressure of at least 15 pounds per square inch (psi), and preferably no more than 30 psi.

In certain embodiments, subjecting the resultant parison to a heat setting process comprises: heating the parison to a temperature greater than or equal to the temperature at which the balloon was axially stretched and radially expanded, but below the melting temperature of the polymeric material of the tubular parison. In contrast to conventional stretch blow molding processes, relatively low pressures are used, which are reduced further during the heat setting process to allow the expanded parison to retract to a profile/size approaching that of the original tubing.

Such process is desirable because the resultant balloon can be inflated to a variety of diameters forming various volumes, as desired.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

As used herein, the term “or” is generally employed in its sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWING

FIGS. 1a-d show the distal end of a balloon catheter incorporating a balloon made by a method of the present invention showing a deflated profile and increased inflated profiles respectfully.

### DETAILED DESCRIPTION OF ILLUSTRATIVE EMBODIMENTS

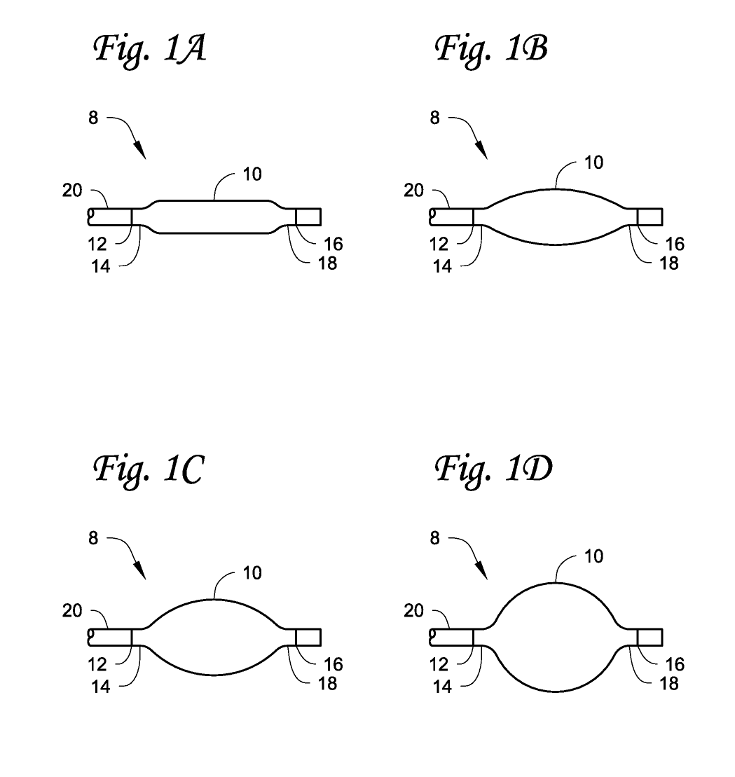

The present invention provides a method of making a zero-fold dilatation balloon. Typically, the method includes: providing a tubular parison comprising a polymeric material; providing a source of heat and pressure for forming a balloon pre-form; heating, stretching, and expanding the tubular parison to form an expanded parison without confining the size of the expanded parison by a mold internal surface; and subjecting the resultant parison to a heat setting process to form a zero-fold balloon having a uniform profile along its entire length in a deflated state as shown in FIG. 1a.

Such a process is desirable because the resultant low pressure compliant balloon can be inflated to a variety of diameters forming various volumes, as desired as shown in the figures. Preferably, such balloons can be inflated up to 15 millimeters (mm) in diameter

Turning now to the figures, balloon catheter 8 includes a balloon body 10 having a proximal end 12 with a proximal neck 14 and a distal end 16 with a proximal neck 18. The balloon body 10 between the ends includes a continuous polymer tube with a uniform profile along its entire length in a deflated state. Balloon 8 is mounted on catheter shaft 20 such that its deflated outer diameter approximates the outer diameter of the catheter shaft.

Materials used in balloons of the present invention are primarily thermoplastics or thermoplastic elastomers. They may be block co-polymers, graft co-polymers, a blend of elastomers and thermoplastics, and the like. Such polymers may be crosslinked or not. Various combinations of polymers may be used in making balloons of the present invention. Exemplary materials include polyesters and copolymers thereof, polyamides and copolymers thereof, polyethylenes and copolymers thereof, and polyurethanes and copolymers thereof. Typically, and preferably, such polymers are block copolymers. Examples of mixtures of polymers include mixtures of nylon and polyamide block copolymers and polyester block copolymers.

Other useful materials include polyesterether and polyetheresteramide copolymers such as those described in U.S. Pat. No. 5,290,306 (Trotta et al.), polyether-polyamide copolymers such as those described in U.S. Pat. No. 6,171,278 (Wang et al.), polyurethane block copolymers such as those described in U.S. Pat. Nos. 6,210,364 B1, 6,283,939 B1, and 5,500,180 (all to Anderson et al.). Suitable polymers also include materials such as the multiblock copolymers of the zero-fold balloon described in U.S. Pat. Pub. No. 2005/0118370.

A particularly preferred block copolymer which can be used in accordance with the process of this invention is polyurethane block copolymer. This preferred polymer may be made, for example, by a reaction between a) an organic diisocyanate; b) a polyol; and c) at least one chain extender. Preferred polyurethanes which can be used in this invention may be varied by using different isocyanates and polyols which will result in different ratios of hard to soft segments as well as different chemical interactions within the individual regions of the polymer. They may include various durometer polyurethanes (e.g., 70a to 95a) available under the trade designations TECOFLEX (Thermedics Polymer Products), PELLETHANE (Dow Chemical Company), and polyether block amide copolymers available under the trade designation PEBAX. Low durometer segmented block copolymers such as TECOFLEX 80a and PELLETHANE 2363 80a are particularly useful.

The balloon tubing is selected to ensure a particular ratio of the balloon diameter and its neck. The ratio determines the range of inflated diameters that could be achieved depending on the neck size and the ratio depends upon the balloon material. In one embodiment using a polyurethane material, a maximum balloon body to neck ratio of about 6 to 1 is preferred. Thus, for a balloon neck of 1 mm diameter, the maximum balloon diameter would be 6 mm; for a balloon neck of 2.5 mm diameter, the maximum balloon diameter would be 15 mm; and for a 5 mm neck diameter, the maximum balloon diameter would be 30 mm. The length of the balloon body (i.e., the continuous polymer tube) is at least 10-25 mm in length, but could range preferably from 6-40 mm.

Balloons of the present invention have a balloon body that includes a continuous polymer tube with a wall thickness that is typically the same throughout. In certain embodiments, the wall thickness of the balloon body is at least 0.1 mm. In certain embodiments, the wall thickness of the balloon body is no more than 0.25 mm.

Balloons of the present invention are zero-fold. The phrase zero-fold is used herein to refer to balloons that have no folds or wraps.

Balloons of the present invention are compliant. This classification is based upon the operating characteristics of the individual balloon, which in turn depend upon the process used in forming the balloon, as well as the material used in the balloon forming process. A balloon which is referred to as being “compliant” is characterized by the balloon's ability to grow or expand beyond its nominal or rated diameter. In balloons currently known in the art (e.g., polyethylene, polyvinylchloride), the balloon's compliant nature or distensibility results from the chemical structure of the polymeric material used in the formation of the balloon, as well as the balloon forming process. Compliant balloons upon subsequent inflations, will achieve diameters which are greater than the diameters which were originally obtained at any given pressure during the course of the balloon's initial inflation. Dimensions provided herein are the dimensions of the balloon when it is in a fully inflated state and at its nominal or rated diameter (i.e., upon initial inflation for a compliant balloon), unless otherwise specified.

Preferred balloons of the present invention have high elasticity and high elastic recovery, which gives rise to self-wrapping characteristics. Self-wrapping refers to the characteristic of a highly elastic balloon where, after initial inflation and upon deflation, the balloon returns to a uniform profile over the catheter tubing. Preferably, the balloon returns to approximately the same profile it had before the initial inflation. To further assist in the elastic recovery, stretching the balloon when bonding it to the catheter shaft offsets the potential for the balloon to become baggy after repeated inflations.

The term “elastic,” as it is used in connection with this invention, refers only to the ability of a material to follow the same stress-strain curve upon the multiple applications of stress. Elasticity, however, is not necessarily a function of how distensible a material is. It is possible to have an elastic, non-distensible material or a non-elastic, distensible material.

Before initial inflation and when deflated, balloons of the present invention preferably have a much lower profile than wrapped conventional balloons, and can have essentially the same dimensions as the tubular pre-form. They preferably revert to the initial tubular form when deflated, even after multiple inflations and after multiple lesions have been dilated. Balloons of the present invention have elasticity at nominal strains of at least 30%. Alternatively, balloons of the present invention have elastic recovery from nominal strains equal to, or greater than, 30%, 40%, 50%, 60%, 70%, 80%, 90%, or 100%, where nominal strain is [(balloon o.d. at nominal pressure-preform o.d.)/preform o.d.]×100, where “o.d.” is the outer diameter. Preferred balloons of the present invention may, therefore, be used to dilate multiple lesions without compromising primary performance.

If desired, balloons of the present invention have a balloon body that includes a continuous polymer tube having a hydrophilic coating thereon to decrease the friction between sliding surfaces. Such hydrophilic coating is typically applied to the continuous polymer tube by coating the hydrophilic material on the continuous polymer tube as is done conventionally in the art. This can be done when the balloon is in the inflated state or in the uninflated state. If necessary, such coating material can be cured using radiation, such as ultraviolet light. Exemplary materials for the hydrophilic coating include PhotoLink® lubricity coating made by SurModics, Inc.

In accordance with this invention, the balloons are formed from a thin wall parison of a polymeric material, preferably made of a polyurethane block copolymer, optionally using a mold, which can be provided with a heating element.

In a preferred embodiment, a mold receives a tubular parison made of a polymeric material, although a mold is not necessary since the mold is not used to confine the expanded parison to the internal surface of the mold. The ends of the parison extend outwardly from the mold and one of the ends is sealed while the other end is affixed to a source of inflation fluid, typically nitrogen gas, under pressure. Clamps or “grippers” are attached to both ends of the parison so that the parison can be drawn apart axially in order to axially stretch the parison while at the same time said parison is capable of being expanded radially or “blown” with the inflation fluid. The radial expansion and axial stretch step or steps may be conducted simultaneously, or depending upon the polymeric material of which the parison is made, following whatever sequence is required to form a balloon. Failure to axially stretch the parison during the balloon forming process will result in a balloon that will have an uneven wall thickness and will exhibit a wall tensile strength lower than the tensile strength obtained when the parison is both radially expanded and axially stretched.

The polymeric parisons used in this invention are preferably drawn axially and expanded radially simultaneously. To improve the overall properties of the balloons formed, it is desirable that the parison is axially stretched and blown at temperatures above the glass transition temperature (Tg) of the polymeric material used. This expansion usually takes place at a temperature of 80° C. to 150° C., depending upon the polymeric material used in the process.

In accordance with this invention, based upon the polymeric material used, the parison is dimensioned with respect to the intended final configuration of the balloon. It is particularly important that the parison have relatively thin walls. The wall thickness is considered relative to the inside diameter of the parison which has a wall thickness-to-inside diameter ratio of less than 0.6, and preferably between 0.57 and 0.33 or even lower. The use of a parison with relatively thin walls enables the parison to be stretched radially to a greater and more uniform degree because there is less stress gradient through the wall from the surface of the inside diameter to the surface of the outside diameter. By utilizing a parison which has thin walls, there is less difference in the degree to which the inner and outer surfaces of the tubular parison are stretched.

Preferably, the parison is drawn from a starting length L1 to a drawn length L2, which preferably is between about 1.10 to about 6 times the initial length L1. The tubular parison, which has an initial internal diameter ID1 and an outer diameter OD1, is expanded by the inflation fluid emitted under pressure to an outer diameter OD2, which is about 1.1 to 2 times the initial outer diameter OD1. The parison is subjected to a cycle during which the parison is axially stretched and radially expanded with a pressure sufficient to stretch and thin the balloon walls, preferably at a pressure of at least 15 pounds per square inch (psi), and preferably no more than 30 psi. Significantly, such pressures are much lower (e.g., at least 10 times lower) than conventional blow molding processes, which are typically carried out at about 300 psi. Nitrogen gas is the preferable inflation fluid for the radial expansion step.

After the forming stage, optionally in a mold but without being confined to the internal surface of the mold, the resultant parison is subjected to a heat setting process. During this process the parison is preferably subjected to a temperature greater than or equal to the temperature at which the balloon was axially stretched and radially expanded, but below the melting temperature of the polymeric material from which the parison was formed. Typically, the temperature chosen is one that induces crystallization and “freezes” or “locks” the orientation of the polymer chains which resulted from axially stretching and radially expanding the parison. The temperatures which can be used in this heat setting step are therefore dependent upon the particular polymeric material used to form the parison and the ultimate properties desired in the balloon product (e.g., distensibility, strength, and compliancy). Preferably, the pressure is reduced during the heat setting step, which causes the parison to retract to the pre-form shape as shown in FIG. 1a.

The heat in the setting process is applied for a time sufficient to form a zero-fold balloon having a uniform profile along its entire length in a deflated state. The heat setting step ensures that the expanded parison and the resulting balloon will have temperature and dimensional stability.

After the heat setting step is completed, the mold is cooled to room temperature.

If the parison is formed from a low durometer segmented block copolymer such as TECOFLEX 80a/PELLETHANE 2363 80a , and axially stretched and radially expanded at a temperature of 90-100° C., the heat set step would preferably be conducted at about 105-120° C. If this step is conducted at temperatures much above 120° C., the tensile strength of the resulting polyurethane balloon would decrease significantly. Moreover, if the heat set step is conducted at temperatures significantly higher than 120° C., the distensibility of the resulting polyurethane balloon would also be adversely affected. However, if the heat set is conducted at temperatures below 100° C., the polyurethane balloons formed would be dimensionally unstable resulting in balloons with uneven wall thicknesses. Additionally, the lower heat set temperature would result in balloons exhibiting physical properties that would more likely be adversely affected during sterilization. Typical sterilization processes used for balloon catheters can be used to sterilize the balloons of the present invention.

### EXAMPLES

Objects and advantages of this invention are further illustrated by the following examples, but the particular materials and amounts thereof recited in these examples, as well as other conditions and details, should not be construed to unduly limit this invention.

The balloon pre-forms were produced via a multi-step forming comprising the following main steps:

a) inserting the tubing through the mold;

b) closing the mold and clamping the tubing;

c) heating, pressurizing, and stretching the tubing;

d) heat setting the balloon pre-form shape; and

e) cooling and releasing the balloon pre-form from the mold

In contrast to conventional blow molding processes, very low pressures were used such as 17.5 psi to about 30 psi. This produced a balloon pre-form shape that inflated as shown in FIG. 1 when subsequently inflated with a syringe. The inflation characteristics of the resultant balloon are similar to those of a dip molded balloon but without having the disadvantage of a low volume production process. Although a mold was used in this particular instance, it is conceived of here that the pre-form could be formed in air or free-formed, since the requirement is only to stretch and pre-shape the tubing rather than conform to a mold surface.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method of making a zero-fold dilatation balloon, the method comprising:
providing a tubular parison comprising a polymeric material;
providing a source of heat and pressure for forming a balloon pre-form;
heating, stretching, and expanding the tubular parison to form an expanded parison without confining the size of the expanded parison by a mold wall internal surface; and
subjecting the resultant parison to a heat setting process carried out for a time sufficient to form a zero-fold balloon having a uniform profile along its entire length in a deflated state.

2. The method of claim 1 wherein expanding the tubular parison to form an expanded parison comprises axially stretching and radially expanding the tubular parison at a temperature above the Tg of the polymeric material.

3. The method of claim 2 wherein expanding the tubular parison to form an expanded parison comprises axially stretching and radially expanding the tubular parison at an inflation pressure of no greater than 30 psi.

4. The method of claim 3 wherein expanding the tubular parison to form an expanded parison comprises axially stretching and radially expanding the tubular parison at an inflation pressure of at least 15 psi.

5. The method of claim 4 wherein subjecting the resultant parison to a heat setting process comprises reducing the inflation pressure during the heat setting step.

6. The method of claim 1 wherein the radial expansion and axial stretch steps are conducted simultaneously.

7. The method of claim 1 subjecting the resultant parison to a heat setting process comprises heating the parison to a temperature greater than or equal to the temperature at which the balloon was axially stretched and radially expanded, but below the melting temperature of the polymeric material of the tubular parison.

8. The method of claim 1 wherein the polymeric material comprises one or more thermoplastic polyurethane polymers.

9. A method of making a zero-fold dilatation balloon, the method comprising:
providing a tubular parison comprising a polymeric material;
providing a source of heat and pressure for forming a balloon pre-form;
axially stretching and radially expanding the tubular parison at a temperature above the Tg of the polymeric material and at a pressure of no greater than 30 psi to form an expanded parison without confining the size of the expanded parison by a mold wall internal surface; and
subjecting the resultant parison to a heat setting process while reducing the inflation pressure, which is carried out for a time sufficient to form a zero-fold balloon having a uniform profile along its entire length in a deflated state;
wherein the heat setting process comprises heating the parison to a temperature greater than or equal to the temperature at which the balloon was axially stretched and radially expanded, but below the melting temperature of the polymeric material of the tubular parison.

10. A zero-fold dilatation balloon prepared by the method of any one of claims 1 through 9.

11. A zero-fold dilatation balloon comprising:
a balloon body having a proximal end and a distal end, and comprises a continuous thermoplastic elastomeric polymer tube with a uniform profile along its entire length in a deflated state;
wherein such balloon can be inflated to various sizes up to 15 mm in diameter.
